(19) United States
(12) Patent Application Publication
(10) Pub. No.: US 2012/0172726 A1
(43) Pub. Date: Jul. 5, 2012

(54) ULTRASONIC DIAGNOSTIC DEVICE

(75) Inventor: Tomohito Sakai, Kanagawa (JP)

(73) Assignee: PANASONIC CORPORATION, Kadoma-shi, Osaka (JP)

(21) Appl. No.: 13/395,699

(22) PCT Filed: Dec. 3, 2010
(86) PCT No.: PCT/JP2010/007045

§ 371 Date: Mar. 13, 2012

(30) Foreign Application Priority Data
Dec. 4, 2009 (JP) 2009-276653

(51) Int. Cl. A61B 8/14

(52) U.S. Cl. 600/443

(57) ABSTRACT

An ultrasonic diagnostic device includes the following: a transmission/reception unit (2) that generates an echo signal from a reception signal transmitted from a probe that receives ultrasonic waves; an image generation unit (3) that generates ultrasonic image data from the echo signal; a display control unit (4) that outputs the ultrasonic image data or synthetic image data obtained by synthesizing various setting items and an ultrasonic image based on the ultrasonic image data; and display unit (5) that displays the ultrasonic image data as an ultrasonic image or the synthetic image data as a synthetic image. The ultrasonic diagnostic device further includes a display switching control unit (6) that analyzes a predetermined switching factor, and outputs a display switching signal to the display control unit (4) when the display switching control unit (6) recognizes that a display switching condition for the switching of an image to be displayed on the display unit (5) between the ultrasonic image and the synthetic image is satisfied. The display control unit (4) switches the image to be displayed on the display unit (5) based on the display switching signal. With this configuration, the operator can switch the display states of the ultrasonic image and the various setting items by performing a normal diagnostic operation.

### TECHNICAL FIELD

The present invention relates to an ultrasonic diagnostic device, and particularly to switching control of an image display state of the ultrasonic diagnostic device.

### BACKGROUND ART

A conventional ultrasonic diagnostic device including a means for switchably displaying a setting image and a diagnostic image on a monitor has been proposed (see, e.g., Patent Document 1). In the conventional ultrasonic diagnostic device, the setting image is obtained by synthesizing an ultrasonic image and various setting items related to the ultrasonic image, and the diagnostic image is obtained by maximizing a display area of the ultrasonic image.

FIG. 7 is a schematic view showing switching between a setting image 101 containing various setting items 103 and a diagnostic image 102. This ultrasonic diagnostic device has an instruction means used for an operator to provide instructions to switch the display states. The instruction means can be a switch key. When the switch key is pressed down, the operator's instructions are input, and then the display states are switched. With this configuration, since the display area of the ultrasonic image can be maximized during examination, the diagnostic performance can be improved. Moreover, the settings of the ultrasonic diagnostic device can be changed by switching the display images.

### Prior Art Documents

### Patent Documents

Patent Document 1: JP 2003-159252A

### DISCLOSURE OF INVENTION

### Problem to be Solved by the Invention

However, in the above conventional configuration, the operator needs to operate the instruction means in some way (e.g., pressing down the switch key) to switch the display states. For this reason, the operation becomes complicated.

The present invention solves the above conventional problem and has an object of providing an ultrasonic diagnostic device that allows an operator to switch the display states of an ultrasonic image and various setting items by performing a normal diagnostic operation.

### Means for Solving Problem

An ultrasonic diagnostic device of the present invention includes the following: a transmission/reception unit that generates an echo signal from a reception signal transmitted from a probe that receives ultrasonic waves; an image generation unit that generates ultrasonic image data (diagnostic image data) from the echo signal; a display control unit that outputs the ultrasonic image data or synthetic image data (setting image data) obtained by synthesizing various setting items and an ultrasonic image based on the ultrasonic image data; and a display unit that displays the ultrasonic image data as an ultrasonic image or the synthetic image data as a synthetic image. To solve the above problem, the ultrasonic diagnostic device further includes a display switching control unit. The display switching control unit analyzes a predetermined switching factor, and outputs a display switching signal to the display control unit when the display switching control unit recognizes that a display switching condition for the switching of an image to be displayed on the display unit between the ultrasonic image and the synthetic image is satisfied. The display control unit switches the image to be displayed on the display unit based on the display switching signal.

In the ultrasonic diagnostic device, the display switching control unit may include a timer that resets an elapsed time, which is the predetermined switching factor, when an operator performs a predetermined operation in a diagnostic operation. The display switching control unit may output the display switching signal indicating display of the ultrasonic image to the display control unit when the elapsed time is not less than a predetermined value as the display switching condition. Moreover, the timer may have an elapsed time setting portion that allows the operator to set the predetermined value of the timer.

The display switching control unit may analyze a signal from a sensor, and output the display switching signal.

The display switching control unit may include a sensor that measures a distance between the ultrasonic diagnostic device and an operator, which is the predetermined switching factor. The display switching control unit may analyze the distance measured by the sensor, and output the display switching signal indicating display of the synthetic image to the display control unit when the distance measured by the sensor is less than a predetermined distance as the display switching condition, and also output the display switching signal indicating display of the ultrasonic image to the display control unit when the distance measured by the sensor is not less than the predetermined distance as the display switching condition.

Moreover, the display switching control unit may include a line-of-sight sensor that detects a line-of-sight of an operator, which is the predetermined switching factor. The display switching control unit may analyze the line-of-sight of the operator detected by the line-of-sight sensor, and output the display switching signal based on the results of the analysis. Moreover, the display switching control unit may include an acceleration sensor that detects a movement of the probe, which is the predetermined switching factor. The display switching control unit may analyze the movement of the probe detected by the acceleration sensor, and output the display switching signal based on the results of the analysis.

The display switching control unit may analyze the echo signal from the transmission/reception unit or the ultrasonic image data from the image generation unit, and output the display switching signal based on the results of the analysis.

The display switching control unit may analyze the echo signal from the transmission/reception unit or the ultrasonic image data from the image generation unit, and output the display switching signal indicating display of the synthetic image to the display control unit when the display switching control unit judges that the ultrasonic waves are radiated through the air as the display switching condition, and also output the display switching signal indicating display of the ultrasonic image to the display control unit when the display switching control unit judges that the ultrasonic waves are not radiated through the air as the display switching condition.

The display switching control unit may include a workflow analysis unit that analyzes an operational history, which is the predetermined switching factor. The display switching control unit may output the display switching signal to the display control unit based on the results of the analysis of the workflow analysis unit. Moreover, the display switching control unit may have an operation setting portion that can set an operation that is analyzed as the display switching condition by the workflow analysis unit.

The display switching control unit may allow an operator to set an operation by which the display switching control unit decides to perform display switching.

### Effects of the Invention

According to the present invention, the ultrasonic diagnostic device includes the display switching control unit for determining the display switching, and thus allows switching of the display states of the ultrasonic image, the various setting items, and the additional information by performing a normal diagnostic operation without any particular operation to change the display states.

### BRIEF DESCRIPTION OF DRAWINGS

FIG. 1 is a block diagram of an ultrasonic diagnostic device of Embodiment 1 of the present invention.

FIG. 2 is a schematic view showing an operation of an ultrasonic diagnostic device of the present invention.

FIG. 3 is a block diagram of an ultrasonic diagnostic device of Embodiment 2 of the present invention.

FIG. 4 is a block diagram of an ultrasonic diagnostic device of Embodiment 3 of the present invention.

FIG. 5 is a block diagram of an ultrasonic diagnostic device of Embodiment 4 of the present invention.

FIG. 6 is a block diagram of an ultrasonic diagnostic device of Embodiment 5 of the present invention.

FIG. 7 is a schematic view showing an operation of a conventional ultrasonic diagnostic device.

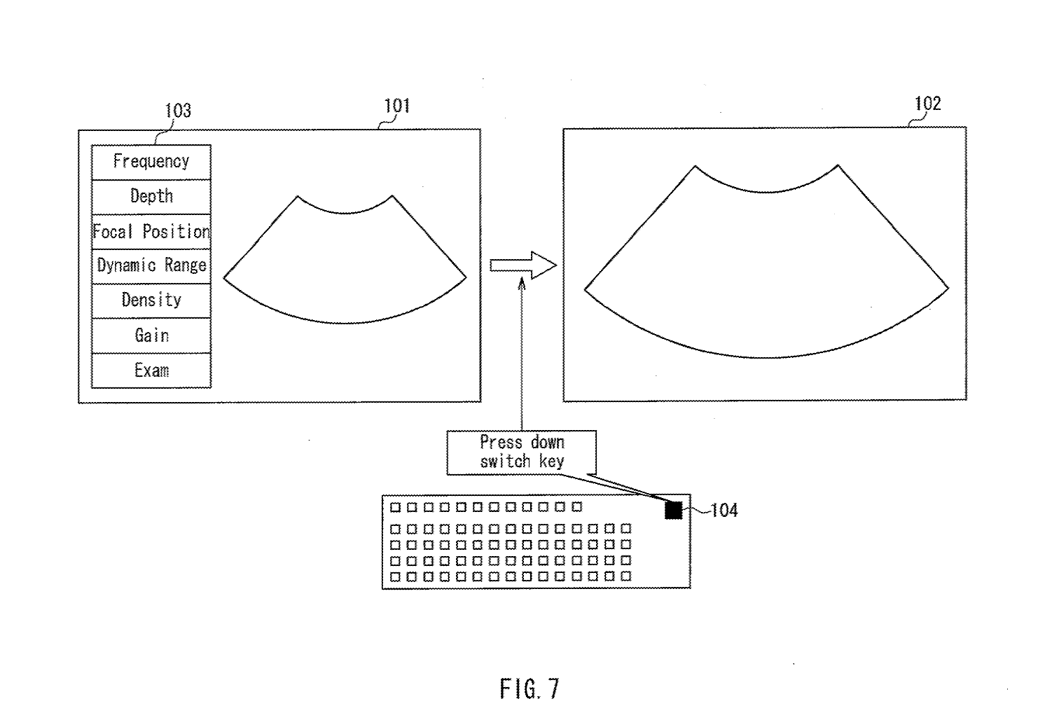

### DESCRIPTION OF THE INVENTION

### Embodiment 1

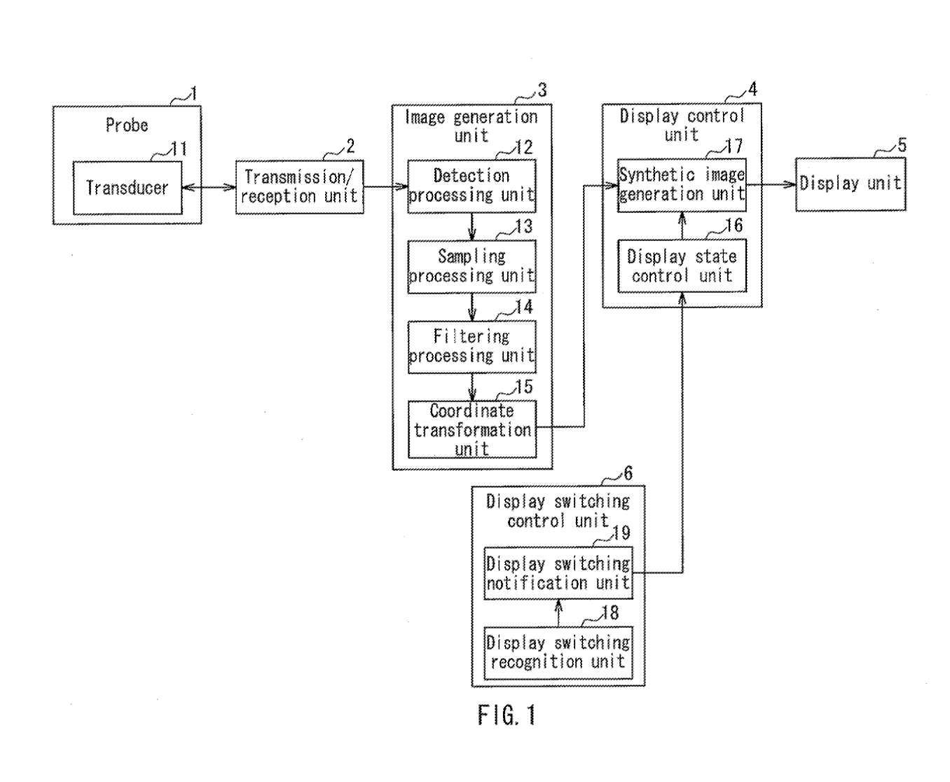

FIG. 1 is a block diagram showing a configuration of an ultrasonic diagnostic device of Embodiment 1 of the present invention. A probe 1 has a transducer 11, and converts a transmission signal (electrical signal) into ultrasonic waves and irradiates a subject body with the ultrasonic waves. Moreover, the probe 1 converts the ultrasonic waves that have been reflected by the interface between materials with different acoustic impedances in the subject body into an electrical signal (reception signal). A transmission/reception unit 2 generates a drive signal for the transducer 11 and controls the delay of the drive signal to generate a transmission signal. The transmission/reception unit 2 also performs delay addition on the reception signal and generates an echo signal from signals for each of the acoustic lines.

An image generation unit 3 includes, e.g., a detection processing unit 12, a sampling processing unit 13, a filtering processing unit 14, and a coordinate transformation unit 15. The image generation unit 3 performs processing such as detection, sampling, filtering, and coordinate transformation on the echo signal and produces ultrasonic image data.

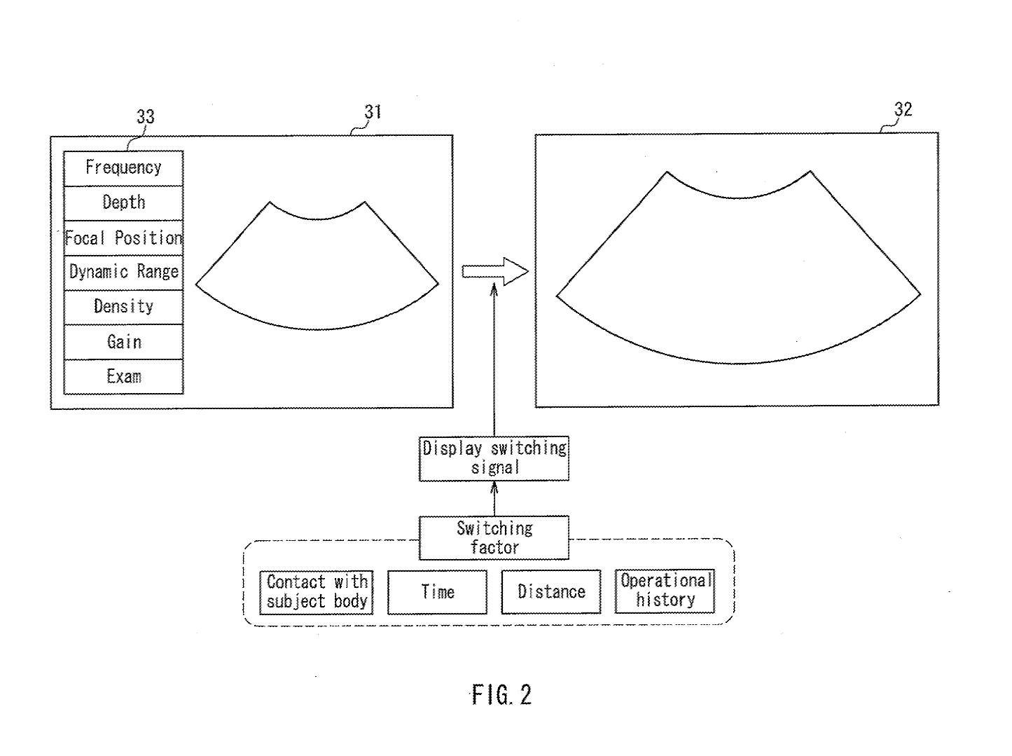

A display control unit 4 includes, e.g., a display state control unit 16 and a synthetic image generation unit 17. The display state control unit 16 receives a display switching signal from a display switching notification unit 19 (as will be described later) and recognizes the display state switching between an image (referred to as “diagnostic image” in the following) obtained by maximizing an ultrasonic image and an image (referred to as “setting image” in the following) obtained by synthesizing the ultrasonic image and various setting items (setting items, additional information, etc.) in accordance with the display switching signal, and then decides whether image synthesis will be required. FIG. 2 is a schematic view showing the display state switching between a setting image 31 containing various setting items 33 and a diagnostic image 32 based on a switching factor.

The display state control unit 16 holds the display position information and the display order information of various display items. The synthetic image generation unit 17 generates diagnostic image data or setting image data based on the display position information and the display order information of various display items in accordance with the decision of the display state control unit 16. A display unit 5 includes, e.g., a monitor. The display unit 5 displays the diagnostic image data as a diagnostic image and the setting image data as a setting image.

A display switching control unit 6 includes, e.g., a display switching recognition unit 18 and the display switching notification unit 19. The display switching control unit 6 determines the display state of a display image that is to be displayed on the display unit 5. The display switching recognition unit 18 determines which display image, the diagnostic image or the setting image, to display on the display unit 5 based on the display switching information in the switching factor. The display switching information is detected inside the display switching recognition unit 18 or externally input to the display switching recognition unit 18. When the display switching information satisfies the display switching condition, the display switching notification unit 19 transmits a display switching signal to the display control unit 4.

The display switching condition may be either set in the device previously or set by an operator. The switching factor can be of any type as long as the display switching information can distinguish the case where the operator is examining the subject body using the probe 1 from the case where the operator is changing the settings. Thus, the display switching control can be performed so that the maximized ultrasonic image is displayed on the display unit 5 during the examination, and both the ultrasonic image and the various setting items are displayed on the display unit 5 at the time of changing the settings of image parameters.

Next, the operation of the ultrasonic diagnostic device having the above configuration of this embodiment will be described. The transmission/reception unit 2 controls the information about the transmission of the ultrasonic waves and transmits a transmission signal to the probe 1. The probe 1 is driven by the transmission signal and transmits ultrasonic waves to the subject body. Moreover, the probe 1 receives the reflected waves from the subject body and transmits a reception signal to the transmission/reception unit 2. The transmission/reception unit 2 performs the delay addition on the reception signal to generate an echo signal and transmits the echo signal to the image generation unit 3. The image generation unit 3 generates ultrasonic image data from the echo signal and transmits the ultrasonic image data to the display control unit 4.

The display switching control unit 6 decides whether the display switching information satisfies the display switching condition, and then transmits a display switching signal to the display control unit 4. The display control unit 4 receives the display switching signal, generates diagnostic image data or setting image data, and transmits the generated data to the display unit 5. The display unit 5 displays the diagnostic image data as the diagnostic image 32 and the setting image data as the setting image 31.

As described above, in this embodiment, the display switching control unit 6 transmits the display switching signal to the display control unit 4 in accordance with the examination situation, and the display control unit 4 switches the image to be displayed on the display unit 5. Therefore, the display states can be switched automatically without the need for the operator consciously to perform a switching operation, and the image with desired content and size can be displayed. Thus, not only the visibility of the image but also the operability is improved, so that the time for diagnosis can be reduced.

### Embodiment 2

Embodiment 2 describes a specific example of the display switching control unit 6 in Embodiment 1. In an ultrasonic diagnostic device of this embodiment, the predetermined switching factor is an elapsed time from a predetermined operation. Moreover, the elapsed time measured by a timer is used as the display switching information. Specifically, the setting image is displayed while the operator is performing a predetermined operation on the ultrasonic diagnostic device. When a predetermined time has elapsed since the predetermined operation, the diagnostic image is displayed, assuming that the operator is examining the subject body at the moment.

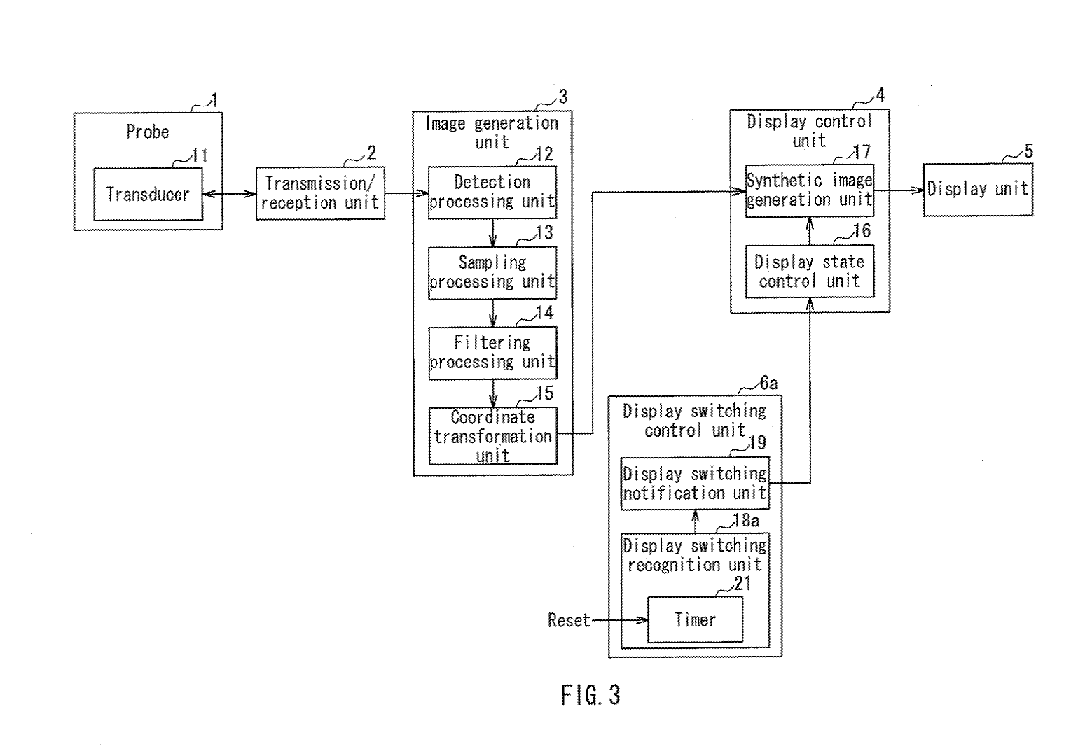

FIG. 3 is a block diagram showing a configuration of the ultrasonic diagnostic device of Embodiment 2 of the present invention. The ultrasonic diagnostic device of this embodiment uses a display switching control unit 6a instead of the display switching control unit 6 of the ultrasonic diagnostic device of Embodiment 1. The other components of the ultrasonic diagnostic device of this embodiment are the same as those of the ultrasonic diagnostic device of Embodiment 1. The same components as those of Embodiment 1 are denoted by the same reference numerals, and the explanation will not be repeated.

A display switching recognition unit 18a of the display switching control unit 6a has a timer 21. The timer 21 measures an elapsed time from the time at which the operator performs any operation on the ultrasonic diagnostic device. The elapsed time measured by the timer 21 corresponds to the display switching information in Embodiment 1. When the operator performs any operation, the timer 21 is reset and starts measuring time again. Thus, the elapsed time from the time of the operation is determined. The display switching recognition unit 18a recognizes that the display switching condition is satisfied when the elapsed time exceeds the previously set time period.

Next, the operation of the ultrasonic diagnostic device having the above configuration of this embodiment will be described. The transmission/reception unit 2 controls the information about the transmission of the ultrasonic waves and transmits a transmission signal to the probe 1. The probe 1 is driven by the transmission signal and transmits ultrasonic waves to the subject body. Moreover, the probe 1 receives the reflected waves from the subject body and transmits a reception signal to the transmission/reception unit 2. The transmission/reception unit 2 performs the delay addition on the reception signal to generate an echo signal and transmits the echo signal to the image generation unit 3. The image generation unit 3 generates ultrasonic image data from the echo signal and transmits the ultrasonic image data to the display control unit 4.

When the elapsed time measured by the timer 21 exceeds the previously set time period, the display switching recognition unit 18a notifies the display switching notification unit 19 that the display switching condition is satisfied. The display switching notification unit 19 transmits a display switching signal indicating the display of the diagnostic image to the display control unit 4. Upon reception of the display switching signal, the display control unit 4 instructs the display unit 5 to change the setting image 31 to the diagnostic image 32.

As described above, in the ultrasonic diagnostic device of this embodiment, the ultrasonic image can be maximized automatically and displayed on the display unit 5, e.g., when the operator has not operated the device for a certain period of time since the previous operation, that is, when the operator has been looking steadily at the image for the diagnosis.

The timer 21 may include an elapsed time setting portion that allows the operator to set a reference time in advance, by which the display switching control unit 6a decides whether the display switching condition is satisfied.

Although the setting image is changed to the diagnostic image in this embodiment, the display switching condition can be set so that the diagnostic image is changed to the setting image. In such a case, an example of the display switching condition is that the operator performs the previously set operation (e.g., pressing the freeze key, the save key, etc.) on the ultrasonic diagnostic device during the examination.

### Embodiment 3

Embodiment 3 describes a specific example of the display switching control unit 6 in Embodiment 1. In an ultrasonic diagnostic device of this embodiment, the predetermined switching factor is a distance from the ultrasonic diagnostic device to the operator. Moreover, the distance measured by a sensor is used as the display switching information. Specifically, when the operator comes close to the main units (the transmission/reception unit 2, the image generation unit 3, the display control unit 4, the display unit 5, and a display switching control unit 6b) of the ultrasonic diagnostic device, the setting image is displayed, assuming that the operator will work on settings. On the other hand, when the operator goes away from the main units of the ultrasonic diagnostic device, the diagnostic image is displayed, assuming that the operator will be examining the subject body.

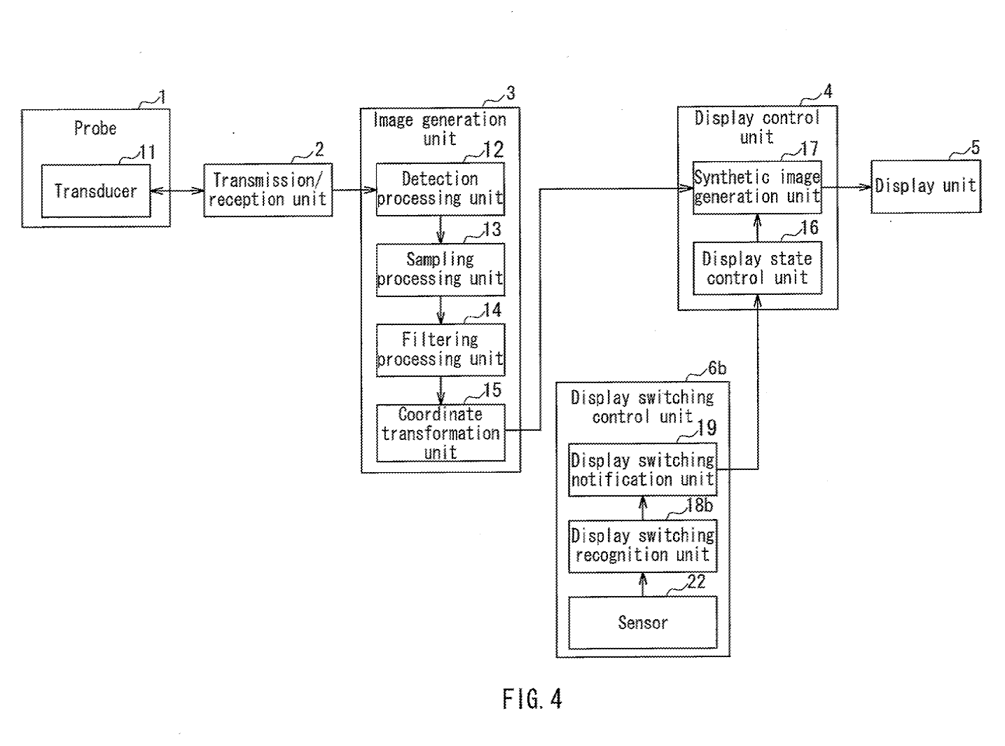

FIG. 4 is a block diagram showing a configuration of the ultrasonic diagnostic device of Embodiment 3 of the present invention. The ultrasonic diagnostic device of this embodiment uses a display switching control unit 6b including a sensor 22 instead of the display switching control unit 6 of the ultrasonic diagnostic device of Embodiment 1. The other components of the ultrasonic diagnostic device of this embodiment are the same as those of the ultrasonic diagnostic device of Embodiment 1. The same components as those of Embodiment 1 are denoted by the same reference numerals, and the explanation will not be repeated.

The sensor 22 measures a distance between any of the main units of the ultrasonic diagnostic device and the operator. The distance measured by the sensor 22 corresponds to the display switching information in Embodiment 1. The display switching control unit 6b includes a display switching recognition unit 18b.

The display switching recognition unit 18b recognizes that the display switching condition for the change from the diagnostic image to the setting image is satisfied when the sensor 22 detects that the operator has entered the area in which the operator can operate the ultrasonic diagnostic device, and then notifies the display switching notification unit 19 of this fact. Moreover, the display switching recognition unit 18b recognizes that the display switching condition for the change from the setting image to the diagnostic image is satisfied when the sensor 22 detects that the operator has moved out of the area in which the operator can operate the ultrasonic diagnostic device, and then notifies the display switching notification unit 19 of this fact.

Next, the operation of the ultrasonic diagnostic device having the above configuration of this embodiment will be described. The transmission/reception unit 2 controls the information about the transmission of the ultrasonic waves and transmits a transmission signal to the probe 1. The probe 1 is driven by the transmission signal and transmits ultrasonic waves to the subject body. Moreover, the probe 1 receives the reflected waves from the subject body and transmits a reception signal to the transmission/reception unit 2. The transmission/reception unit 2 performs the delay addition on the reception signal to generate an echo signal and transmits the echo signal to the image generation unit 3. The image generation unit 3 generates ultrasonic image data from the echo signal and transmits the ultrasonic image data to the display control unit 4.

When the sensor 22 detects that the operator has entered the area in which the operator can operate the ultrasonic diagnostic device, the display switching recognition unit 18b notifies the display switching notification unit 19 that the setting image is to be displayed. The display switching notification unit 19 transmits a display switching signal indicating the display of the setting image to the display control unit 4. Upon reception of the display switching signal, the display control unit 4 instructs the display unit 5 to change the diagnostic image to the setting image.

When the sensor 22 detects that the operator has moved out of the area in which the operator can operate the ultrasonic diagnostic device, the display switching recognition unit 18b notifies the display switching notification unit 19 that the diagnostic image is to be displayed. The display switching notification unit 19 transmits a display switching signal indicating the display of the diagnostic image to the display control unit 4. Upon reception of the display switching signal, the display control unit 4 instructs the display unit 5 to change the setting image to the diagnostic image.

As described above, in the ultrasonic diagnostic device of this embodiment, the maximization of the ultrasonic image can be removed and the diagnostic image can be switched automatically to the setting image, e.g., when the operator stops the scanning operation and is going to change various settings. Moreover, the setting image can be switched automatically to the diagnostic image by maximizing the ultrasonic image, e.g., when the operator performs the scanning operation. Therefore, the operator can examine the subject body without any need to consciously change the settings of the display image. The sensor is not limited to the distance measuring sensor of this embodiment as long as it can detect changes associated with the action of the operator, and may be a line-of-sight sensor for detecting a line of sight of the operator, an acceleration sensor for detecting a movement of the probe, or the like.

In the case of using the line-of-sight sensor for detecting the line of sight of the operator, the display switching control unit 6b analyzes the direction and position of the line of sight of the operator (i.e., the predetermined switching factor) detected by the line-of-sight sensor, and the display switching can be performed based on the results of the analysis. For example, the display can be switched to the diagnostic image when the operator's line of sight has been moved closer to the center of the display screen of the display unit 5. On the other hand, the display can be switched to the setting image when the operator's line of sight has been moved closer to the edge of the display image or moved away from the display screen of the display unit 5. In the case of using the acceleration sensor for detecting the movement of the probe, the display switching control unit 6b analyzes the movement of the probe (i.e., the predetermined switching factor) detected by the acceleration sensor, and can output a display switching signal based on the results of the analysis of the speed, direction, etc. of the movement of the probe.

### Embodiment 4

Embodiment 4 describes a specific example of the display switching control unit 6 in Embodiment 1. In an ultrasonic diagnostic device of this embodiment, the predetermined switching factor is whether the probe is in contact with the subject body. Moreover, the detected information on the presence or absence of contact between the probe and the subject body is used as the display switching information. Specifically, when the operator removes the probe 1 from the subject body to make settings, the setting image is displayed, assuming that the operator will perform a setting operation. On the other hand, when the operator places the probe 1 in contact with the subject body, the diagnostic image is displayed, assuming that the operator will perform a diagnostic operation.

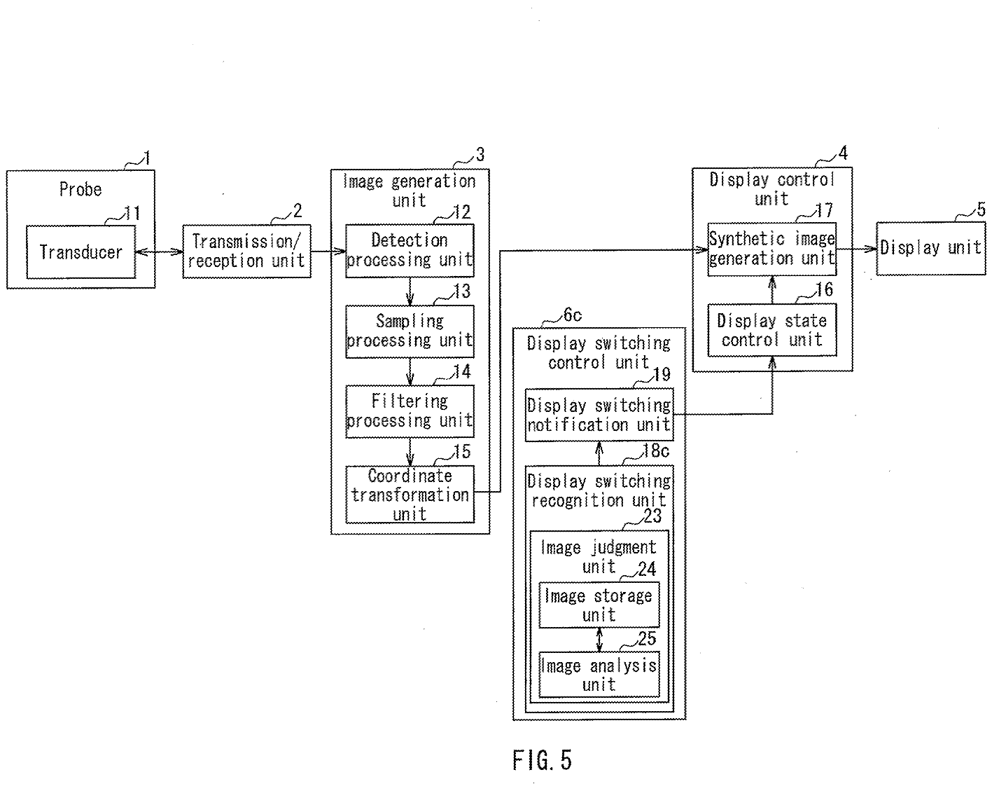

FIG. 5 is a block diagram showing a configuration of the ultrasonic diagnostic device of Embodiment 4 of the present invention. The ultrasonic diagnostic device of this embodiment uses a display switching control unit 6c instead of the display switching control unit 6 of the ultrasonic diagnostic device of Embodiment 1. The other components of the ultrasonic diagnostic device of this embodiment are the same as those of the ultrasonic diagnostic device of Embodiment 1. The same components as those of Embodiment 1 are denoted by the same reference numerals, and the explanation will not be repeated.

A display switching recognition unit 18c of the display switching control unit 6c has an image judgment unit 23 that analyzes the echo data from the transmission/reception unit 2. The image judgment unit 23 includes, e.g., an image storage unit 24 and an image analysis unit 25, and outputs a display switching signal.

The image storage unit 24 stores the echo data from the transmission/reception unit 2 in chronological order. The image analysis unit 25 reads the echo data from the image storage unit 24 in chronological order. Based on the echo data from the transmission/reception unit 2, the image analysis unit 25 analyzes whether the ultrasonic waves emitted from the probe 1 are radiated through the air. The results of the analysis correspond to the display switching information in Embodiment 1. The state where the ultrasonic waves are radiated through the air means that the probe 1 is separated from the subject body. When the echo signal is obtained from the ultrasonic waves that are radiated through the air, the signals for each of the acoustic lines are attenuated uniformly in the depth direction. In other words, the image analysis unit 25 analyzes whether the signals for each of the acoustic lines are attenuated uniformly in the depth direction, and thus can judge whether the ultrasonic waves are radiated through the air.

The display switching recognition unit 18c recognizes that the display switching condition for the change from the diagnostic image to the setting image is satisfied when the image judgment unit 23 detects radiation of the ultrasonic waves through the air, and then notifies the display switching notification unit 19 of this fact. The display switching notification unit 19 transmits a display switching signal indicating the display of the setting image to the display control unit 4. Moreover, the display switching recognition unit 18c recognizes that the display switching condition for the change from the setting image to the diagnostic image is satisfied when the image judgment unit 23 detects that there is no radiation of the ultrasonic waves through the air, and then notifies the display switching notification unit 19 of this fact. The display switching notification unit 19 transmits a display switching signal indicating the display of the diagnostic image to the display control unit 4.

Next, the operation of the ultrasonic diagnostic device having the above configuration of this embodiment will be described. The transmission/reception unit 2 controls the information about the transmission of the ultrasonic waves and transmits a transmission signal to the probe 1. The probe 1 is driven by the transmission signal and transmits ultrasonic waves to the subject body. Moreover, the probe 1 receives the reflected waves from the subject body and transmits a reception signal to the transmission/reception unit 2. The transmission/reception unit 2 performs the delay addition on the reception signal to generate an echo signal and transmits the echo signal to the image generation unit 3. The image generation unit 3 generates ultrasonic image data from the echo signal and transmits the ultrasonic image data to the display control unit 4.

When the operator interrupts the scanning operation and removes the probe 1 from the subject body, the probe 1 receives the reflected waves of the ultrasonic waves that are radiated through the air. The transmission/reception unit 2 converts the reception signal into an echo signal, and the echo signal is stored in the image storage unit 24. The image analysis unit 25 judges that the echo signal is obtained from the ultrasonic waves that are radiated through the air. Then, the display switching recognition unit 18c notifies the display switching notification unit 19 that the display switching condition for the display of the setting image is satisfied. The display switching notification unit 19 transmits a display switching signal indicating the display of the setting image to the display control unit 4. Upon reception of the display switching signal, the display control unit 4 instructs the display unit 5 to change the diagnostic image to the setting image.

When the operator restarts the scanning operation and places the probe 1 in contact with the subject body, the probe 1 receives the reflected waves that are reflected from the subject body. The transmission/reception unit 2 converts the reception signal into an echo signal, and the echo signal is stored in the image storage unit 24. The image analysis unit 25 judges that the echo signal is obtained while the subject body is being examined. Then, the display switching recognition unit 18c notifies the display switching notification unit 19 that the display switching condition for the display of the diagnostic image is satisfied. The display switching notification unit 19 transmits a display switching signal indicating the display of the diagnostic image to the display control unit 4. Upon reception of the display switching signal, the display control unit 4 instructs the display unit 5 to change the setting image to the diagnostic image.

As described above, the ultrasonic diagnostic device of this embodiment makes a judgment as to whether the operator places the probe 1 in contact with the subject body. Consequently, the diagnostic image is displayed when the probe 1 is in contact with the subject body, and the setting image is displayed when the probe 1 is not in contact with the subject body. Therefore, the operator can examine the subject body without any need to consciously change the settings of the display image.

In this embodiment, since the contact of the probe 1 with the subject body is used as the predetermined switching factor, the image judgment unit 23 performs the image analysis using the echo signal from the transmission/reception unit 2. However, the image judgment unit 23 may perform the image analysis using the ultrasonic image data from the image generation unit 3.

In this embodiment, the display switching is performed by judging whether the ultrasonic waves are radiated through the air. However, the display switching condition is not particularly limited as long as it can be determined using the echo signal or the ultrasonic image data.

### Embodiment 5

Embodiment 5 describes a specific example of the display switching control unit 6 in Embodiment 1. In an ultrasonic diagnostic device of this embodiment, the predetermined switching factor is an operational history. Moreover, the results of workflow analysis based on the operational history are used as the display switching information. Specifically, the display switching between the diagnostic image and the setting image is controlled by estimating whether the operator is examining the subject body or setting the ultrasonic diagnostic device based on the results of the workflow analysis.

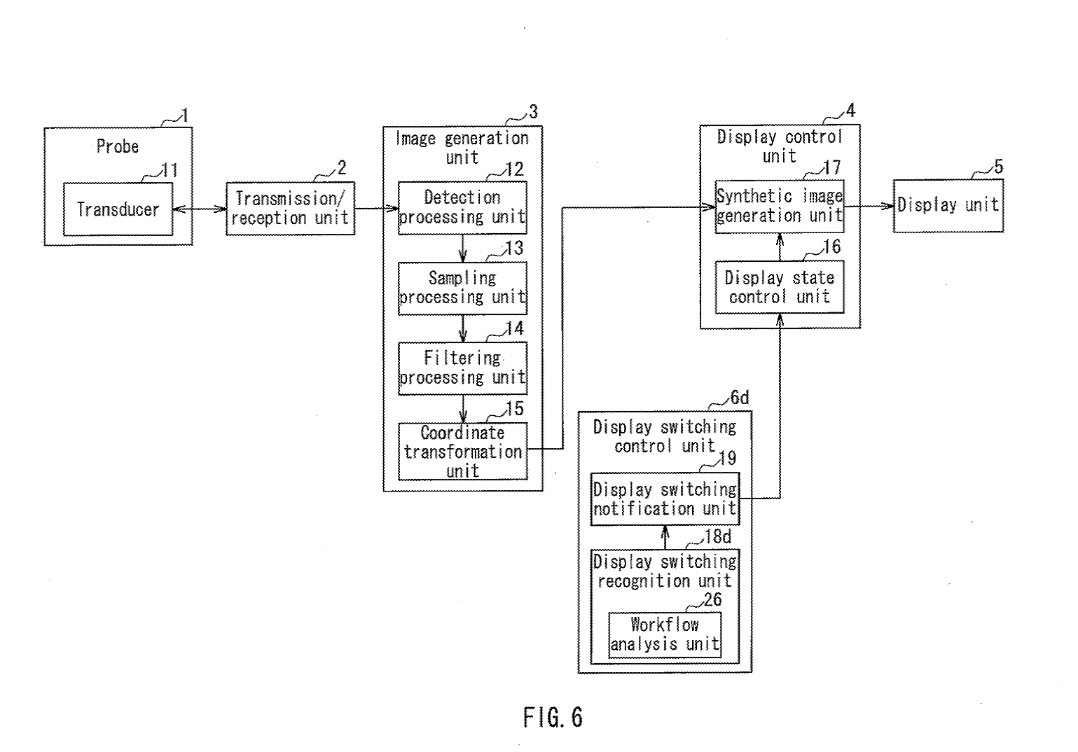

FIG. 6 is a block diagram showing a configuration of the ultrasonic diagnostic device of Embodiment 5 of the present invention. The ultrasonic diagnostic device of this embodiment uses a display switching control unit 6d instead of the display switching control unit 6 of the ultrasonic diagnostic device of Embodiment 1. The other components of the ultrasonic diagnostic device of this embodiment are the same as those of the ultrasonic diagnostic device of Embodiment 1. The same components as those of Embodiment 1 are denoted by the same reference numerals, and the explanation will not be repeated.

A display switching recognition unit 18d of the display switching control unit 6d has a workflow analysis unit 26. The workflow analysis unit 26 receives the operational history of the operator anytime from a history detection unit (not shown) and decides whether the predetermined operation has been performed. The history detection unit is included in a system control unit (not shown). The system control unit operates the other units constituting the system based on all the operations performed by the operator. Moreover, the system control unit recognizes the operations of the operator and transmits the contents of the operations (or, e.g., the operation ID indicating the contents) to the workflow analysis unit 26.

Based on the results of the analysis of the operational history, the workflow analysis unit 26 detects, e.g., a state where “the operator has not performed an operation for a few minutes after changing the gain”, and decides whether the operator has started or ended the scanning operation. The display switching recognition unit 18d notifies the display switching notification unit 19 of the results of the decision, namely the fact that the display switching condition is satisfied.

Next, the operation of the ultrasonic diagnostic device having the above configuration of this embodiment will be described. The transmission/reception unit 2 controls the information about the transmission of the ultrasonic waves and transmits a transmission signal to the probe 1. The probe 1 is driven by the transmission signal and transmits ultrasonic waves to the subject body. Moreover, the probe 1 receives the reflected waves from the subject body and transmits a reception signal to the transmission/reception unit 2. The transmission/reception unit 2 performs the delay addition on the reception signal to generate an echo signal and transmits the echo signal to the image generation unit 3. The image generation unit 3 generates ultrasonic image data from the echo signal and transmits the ultrasonic image data to the display control unit 4.

When the workflow analysis unit 26 analyzes the operational history and decides that the operator has started the scanning operation, i.e., recognizes that the display switching condition is satisfied, the display switching recognition unit 18d notifies the display switching notification unit 19 that the display switching condition for the display of the diagnostic image is satisfied. The display switching notification unit 19 transmits a display switching signal indicating the display of the diagnostic image to the display control unit 4. Upon reception of the display switching signal, the display control unit 4 instructs the display unit 5 to change the setting image to the diagnostic image.

Moreover, when the workflow analysis unit 26 analyzes the operational history and decides that the operator has ended the scanning operation, i.e., recognizes that the display switching condition is satisfied, the display switching recognition unit 18d notifies the display switching notification unit 19 that the display switching condition for the display of the setting image is satisfied. The display switching notification unit 19 transmits a display switching signal indicating the display of the setting image to the display control unit 4. Upon reception of the display switching signal, the display control unit 4 instructs the display unit 5 to change the diagnostic image to the setting image.

As described above, the ultrasonic diagnostic device of this embodiment analyzes the diagnostic operation performed by the operator, and therefore can switch between the diagnostic image and the setting image without the need for the operator to consciously change the settings. Thus, not only the visibility of the image but also the operability is improved, so that the time for diagnosis can be reduced.

In this embodiment, the workflow analysis unit 26 decides whether the particular operation has been performed. However, the display state may be switched based on another operation such as the detection of time. For example, the workflow of diagnosis is input in advance, and the display image can be switched at a predetermined time.

Alternatively, the operator may set the operation by which the workflow analysis unit 26 decides to perform the display switching.

Each of the above Embodiments 1 to 5 provides an overview of the configuration and operation of the ultrasonic diagnostic device by changing the various switching factors for generating the display switching signal in the display switching control unit (see FIG. 2). The display switching factor may be either a single type such as a signal from the timer or the sensor, or a combination of two or more types of these switching factors. In each of the above embodiments, the diagnostic image and the setting image are switched. However, other types of images also can be switched.

### INDUSTRIAL APPLICABILITY

The present invention has the effect of switching the display states of the ultrasonic image and the various setting items without the need for the operator to consciously perform an operation, and is useful as an ultrasonic diagnostic device that places importance on visibility, operability, and a reduction in time for diagnosis.

### DESCRIPTION OF REFERENCE NUMERALS

1 Probe

2 Transmission/reception unit

3 Image generation unit

4 Display control unit

5 Display unit

6, 6a-6d Display switching control unit

11 Transducer

12 Detection processing unit

13 Sampling processing unit

14 Filtering processing unit

15 Coordinate transformation unit

16 Display state control unit

17 Synthetic image generation unit

18, 18a-18d Display switching recognition unit

19 Display switching notification unit

21 Timer

22 Sensor

23 Image judgment unit

24 Image storage unit

25 Image analysis unit

26 Workflow analysis unit

31 Setting image

32 Diagnostic image

33 Various setting items

1. An ultrasonic diagnostic device comprising:
a transmission/reception unit that generates an echo signal from a reception signal transmitted from a probe that receives ultrasonic waves;
an image generation unit that generates ultrasonic image data from the echo signal;
a display control unit that outputs the ultrasonic image data or synthetic image data obtained by synthesizing various setting items and an ultrasonic image based on the ultrasonic image data; and
a display unit that displays the ultrasonic image data as an ultrasonic image or the synthetic image data as a synthetic image,
wherein the ultrasonic diagnostic device further comprises a display switching control unit that analyzes a predetermined switching factor, and outputs a display switching signal to the display control unit when the display switching control unit recognizes that a display switching condition for switching of an image to be displayed on the display unit between the ultrasonic image and the synthetic image is satisfied, and
wherein the display control unit switches the image to be displayed on the display unit based on the display switching signal.

2. The ultrasonic diagnostic device according to claim 1, wherein the display switching control unit includes a timer that resets an elapsed time, which is the predetermined switching factor, when an operator performs a predetermined operation in a diagnostic operation, and
the display switching control unit outputs the display switching signal indicating display of the ultrasonic image to the display control unit when the elapsed time is not less than a predetermined value as the display switching condition.

3. The ultrasonic diagnostic device according to claim 2, wherein the timer has an elapsed time setting portion that allows the operator to set the predetermined value of the timer.

4. The ultrasonic diagnostic device according to claim 1, wherein the display switching control unit analyzes a signal from a sensor, and outputs the display switching signal.

5. The ultrasonic diagnostic device according to claim 4, wherein the display switching control unit includes a sensor that measures a distance between the ultrasonic diagnostic device and an operator, which is the predetermined switching factor, and
the display switching control unit analyzes the distance measured by the sensor, and outputs the display switching signal indicating display of the synthetic image to the display control unit when the distance measured by the sensor is less than a predetermined distance as the display switching condition, and also outputs the display switching signal indicating display of the ultrasonic image to the display control unit when the distance measured by the sensor is not less than the predetermined distance as the display switching condition.

6. The ultrasonic diagnostic device according to claim 4, wherein the display switching control unit includes a line-of-sight sensor that detects a line-of-sight of an operator, which is the predetermined switching factor, and
the display switching control unit analyzes the line-of-sight of the operator detected by the line-of-sight sensor, and outputs the display switching signal based on results of the analysis.

7. The ultrasonic diagnostic device according to claim 4, wherein the display switching control unit includes an acceleration sensor that detects a movement of the probe, which is the predetermined switching factor, and
the display switching control unit analyzes the movement of the probe detected by the acceleration sensor, and outputs the display switching signal based on results of the analysis.

8. The ultrasonic diagnostic device according to claim 1, wherein the display switching control unit analyzes the echo signal from the transmission/reception unit or the ultrasonic image data from the image generation unit, and outputs the display switching signal based on results of the analysis.

9. The ultrasonic diagnostic device according to claim 8, wherein the display switching control unit analyzes the echo signal from the transmission/reception unit or the ultrasonic image data from the image generation unit, and outputs the display switching signal indicating display of the synthetic image to the display control unit when the display switching control unit judges that the ultrasonic waves are radiated through air as the display switching condition, and also outputs the display switching signal indicating display of the ultrasonic image to the display control unit when the display switching control unit judges that the ultrasonic waves are not radiated through air as the display switching condition.

10. The ultrasonic diagnostic device according to claim 1, wherein the display switching control unit includes a workflow analysis unit that analyzes an operational history, which is the predetermined switching factor, and
the display switching control unit outputs the display switching signal to the display control unit based on results of the analysis of the workflow analysis unit.

11. The ultrasonic diagnostic device according to claim 10, wherein the display switching control unit has an operation setting portion that can set an operation that is analyzed as the display switching condition by the workflow analysis unit.

12. The ultrasonic diagnostic device according to claim 1, wherein the display switching control unit allows an operator to set an operation by which the display switching control unit decides to perform display switching.
